(19) United States
(12) Patent Application Publication
(10) Pub. No.: US 2012/0225022 A1
(43) Pub. Date: Sep. 6, 2012

(54) DENTAL CLEANING COMPOSITION FOR ORTHODONTIC PATIENTS

(75) Inventors: Lawrence A. Hier, Boca Raton, FL (US); Ben Vaziri, Mount Prospect, IL (US)

(73) Assignee: SLH Optimal Health LLC, Boca Raton, FL (US)

(21) Appl. No.: 13/358,457

(22) Filed: Jan. 25, 2012

Related U.S. Application Data
(63) Continuation-in-part of application No. 13/013,441, filed on Jan. 25, 2011.
(60) Provisional application No. 61/511,404, filed on Jul. 25, 2011.

(51) Int. Cl. A61K 49/00

(52) U.S. Cl. 424/9.71

(57) ABSTRACT

The present invention relates to oral compositions for identifying plaque comprising a dental disclosing agent composition for revealing the presence of dental plaque in a person's mouth including natural colorants derived from purple carrot.

### RELATED APPLICATIONS

This application claims the benefit under 35 U.S.C. §120 of U.S. application Ser. No. 13/013,441, entitled “DENTAL CLEANING COMPOSITION FOR EVIDENCING DENTAL PLAQUE” filed on Jan. 25, 2011, which is herein incorporated by reference in its entirety. This application claims the benefit under 35 U.S.C. §119 of U.S. Application Ser. No. 61/511,404, entitled “DENTAL CLEANING COMPOSITION FOR ORTHODONTIC PATIENTS” filed on Jul. 25, 2011, which is herein incorporated by reference in its entirety.

### FIELD OF INVENTION

This invention relates generally to plaque disclosing agents and also to dental cleaning compositions.

### BACKGROUND ART

It is a well accepted fact that dental plaque when allowed to accumulate on tooth surfaces can eventually lead to gingivitis, periodontal disease, caries and calculus. Thus, it is apparent that effective removal of deposits of dental plaque is absolutely essential for oral health. Accordingly, a proper oral hygiene practice which may be carried out by an individual on his or her own teeth or by a dentist, necessitates readily available means of identification and location of plaque deposits in the oral cavity. Since dental plaque is usually transparent and colorless and not easily visible, an individual frequently is not aware of the quantity or the location of dental plaque present in the mouth.

The prior art includes various dentifrices for removing plaque, including dentifricating paste, powders, and microbial liquids. As a general matter, these dentrifices contain a mixture of various ingredients including such materials as polishing agents and abrasives for scouring and scrubbing the teeth, and which are further operable, to some degree, to neutralize various acids present in the gaps between the teeth. These same substances further inhibit, to some extent, the subsequent growth of various forms of bacteria that contribute to the development of caries and other disorders. While the prior art dentifrices have varying degrees of success, they have not been successful in arresting decalcification and other diseases which are exasperated by the use of braces.

The amount of decalcification and tooth decay found in orthodontic patients, in fact, is alarming. Numerous studies have been conducted showing the severity of this problem in the orthodontic patient population. Each year, half of the three to five million yearly patients who get braces in the United States suffer from early tooth decay. At present, approximately one in every two patients have enamel decalcification upon removal of their fixed orthodontic appliances. Despite proper oral hygiene instruction by the orthodontist and staff, as well as the presentation to the patient of various dental care aids, this level remains unchanged.

Accordingly, dye indicators for dental plaque as a means of measuring tooth cleanliness and to effect proper oral hygiene practices, have been widely explored in the prior art.

U.S. publication 2007 0237726 ('726 publication”) teaches plaque disclosing products containing coloring agents or pigments that are absorbed by the plaque and render it visible. According to the '726 publication: “Most plaque disclosing compositions are based on colorants such as disclosed in U.S. Pat. Nos. 3,309,274; 3,624,219; 3,997,658; 4,302,439; 4,459,277; 4,517,172; 4,590,061; 4,666,700; 4,992,256; 5,098,691; 5,190,743; 7,182,935. Examples include synthetic organic colorants such as, amongst others, erythrosin (FD&C Red #3), Allura Red (FD&C Red #40), Green #8, Red #19, Red #22, Red #28, fluorescein (Yellow #7) and fluorescein disodium salt (Yellow #8).

The 726 publication teaches that natural colorants have been used as plaque disclosing agents, including a red dye extracted from sugar beet, a salt of sanguinarine, and cobalamin compounds, particularly cyanobalamin (Vitamin B12). According to the '726 publication, some of these colorants are invisible to the human eye in normal daylight or artificial light and may require the use of light of a particular wavelength to become visible.

U.S. Pat. No. 7,182,935 also discloses that natural colorants have been suggested as alternatives to the use of synthetic organic colorants as plaque disclosing agents. According to the '935 patent, artificial colorants have disadvantages which natural colorants do not present. “Some artificial colorants provoke diseases of the thyroid, lesions of the liver, hyperacidity and allergies such as, for example, asthma, rhinitis and rashes.”

According to the '935 patent, examples of natural colorants used to disclose bacterial plaque are taught in U.S. Pat. No. 4,431,628 and U.S. Pat. No. 4,517,172. “The U.S. Pat. No. 4,431,628 refers to a method for indicating the presence of bacterial plaque, comprising an efficient quantity of natural colorant extracted from sugar beet . . . . U.S. Pat. No. 4,517,172 describes a method for the visualization of plaque, in such a manner that the plaque is visible to the naked eye under ultraviolet light. The method employs a salt of sanguinarine precipitated from extracts of plants selected from the group consisting of Sanguinaria canadensis, Macleaya cordata, Corydalis sevctvozii, C. ledebouni, Chelidonium majus and mixtures of these.”

The '935 patent itself is directed to a disclosing agent based on natural colorants comprising at least one concentrated solution of natural colorant selected from the group consisting of colorants extracted from the aai (Euterpe oleracea) and colorants extracted from urucum (Bixa orellana).

It is believed that none of these prior art natural colorants have been commercially successful. It is believed that the reason for the lack of success is that these coloring agents are not visible enough, concentrated enough, and/or stable enough to comprise an effective product. It would be desirable to have a natural colorant that can be used safely and effectively in a dentifrice identifying plaque without the need for a UV light source.

### SUMMARY OF THE INVENTION

The present invention relates to oral compositions for identifying plaque located in a person's mouth comprising a natural dental disclosing agent composition for revealing the presence of dental plaque in a person's mouth. It has been discovered, unpredictably, that a natural colorant in purple carrots is superior to other natural colorants in disclosing plaque. The inventors tested more than forty different dye substances for their capacity to disclose plaque. Among those tested were natural colorants. Only the natural colorant derived from purple carrots had the capacity to disclose plaque at acceptable levels in a patient's mouth. Surprisingly, the natural colorant found in purple carrots could be added to a dental composition, such as a tooth-paste, and still disclose plaque at acceptable levels in a patient's mouth. Finally, in the presence of appropriate preservatives, the natural colorant found in purple carrots was surprisingly stable in dental compositions, such as tooth-paste, making it suitable for commercial application.

Without wishing to be bound by any theory of the invention, it is believed that the quality and concentration of natural colorants in purple carrots is superior to that of other natural colorants for disclosing plaque.

Thus, according to one aspect of the invention, an oral compositions for identifying plaque located in a person's mouth is provided. The composition comprises the natural colorant in purple carrots. In one embodiment, the disclosing agent is a purple carrot extract. In one embodiment, the purple carrot extract is prepared by spray drying. In one embodiment, the purple carrot extract comprises by weight approximately equal parts carbohydrate and sugar. In one embodiment, the purple carrot extract comprised less than 1 percent (1%) by weight protein. In one embodiment, the purple carrot extract consists by weight approximately equal parts carbohydrate and sugar, and contains less than 1 percent (1%) by weight protein. In one embodiment, the purple carrot extract has a color strength in a 1% aqueous solution (E1%, 1% by weight) of 11.5-12.5 at a wave length of 425.

The disclosing agent may optionally include one or more additional natural colorants, including for example, the natural colorant of beets, pomegranates, tomatoes. Such natural colorants can be extracts or concentrates, such as a red beet extract, a pomegranate concentrate, and a tomato extract (e.g., lycopene). In one embodiment, the disclosing agent includes natural colorants derived from a purple carrot extract and a red beet extract. In one embodiment, the disclosing agent includes natural colorants derived from a purple carrot extract and a tomato. In one embodiment the disclosing agent comprises natural colorants derived from a purple carrot extract and pomegranate extract.

In any of the foregoing embodiments, the disclosing agent may be a component of a dental disclosing agent delivery composition, such as tooth paste, for delivering the dental disclosing agent to a person's mouth.

In any of the foregoing embodiments, the oral compositions for identifying plaque located in a person's mouth can comprise a stannous fluoride. Such compositions are particularly beneficial for use by orthodontic patients. The present invention thus provides a method for effective delivery of stannous-containing compositions with effective tartar control by administering to a subject a stable dentifrice composition comprising a clinically effective amount of stannous fluoride and/or other stannous salts in combination with a disclosing substance to highlight areas to be cleaned.

It is believed that the oral compositions of the invention are safe to use and prevent possible harm associated with using synthetic colorants as a source of dental disclosing agents.

In one embodiment, the oral composition is for identifying plaque located in a person's mouth which significantly reduces the amount of decalcification in orthodontic patients.

It is believed that the time involved for any patient is dental care will be greatly reduced, resulting in better compliance and success for the patients.

One aspect of the invention is a composition and a method of treatment for delivering a dental disclosing agent to a person's mouth that lets the patient see, immediately while brushing, where there is plaque build-up on their teeth. Once the stain is removed during the brushing process, the teeth are clean and the risk of decalcification is minimized. In one embodiment, the invention combines a significant concentration of stannous fluoride (which increases the strength of the tooth enamel and reduces the risk of decay), in conjunction with a natural colorant disclosing agent that lets the patient see immediately while brushing, where there is plaque build-up on their teeth. Once the stain is removed during the brushing process, the teeth are clean and the risk of decalcification is minimized.

In one aspect, the invention allows the patient to visually determine the amount of brushing necessary in view of the dental abrasive employed to maximize cleaning and minimizing dentin abrasion.

These and other features, aspects, and advantages of the present invention will become evident to those skilled in the art from the detailed description which follows.

### DETAILED DESCRIPTION OF THE PREFERRED EMBODIMENT

This invention is an oral composition, in one aspect in the form of a toothpaste, that combines a dental disclosing agent composition for revealing the presence of dental plaque in a person's mouth including natural colorants derived from a purple carrot, and a dental disclosing agent delivery composition for delivering the dental disclosing agent to a person's mouth that lets the patient see immediately while brushing, where there is plaque build-up on their teeth. Once the stain is removed during the brushing process, the teeth are clean and the risk of decalcification is minimized. This product will replace any other toothpaste the patient has been using, and is excellent for the entire family to use daily.

The principal disclosing agent in the composition is an extract from purple carrots. One such extract is available from Roha USA, LLC, St. Louis, Mo., 63110. This extract is a spray dried product and is sold under the tradename Nutrcol Purple Carrot Extract. Other disclosing agents which can be combined with the purple carrot to provide the ideal signal when contacted with plaque are known in the art and include the use various types of dyes. A natural red dye from sugar beets is disclosed in U.S. Pat. No. 4,431,628. U.S. Pat. No. 7,182,935 disclosures a plaque evidencing composition using natural colorants extracted from acai and urucum. Organic dyes such as erythrosin as disclosed in U.S. Pat. No. 3,309,274 by Brilliant which utilizes the fluorescent synthetic dyes, FDC colors Red #3, Green #8, Red #19, Red #22, Red #28, Yellow #7 and Yellow #8, which are invisible to the naked eye under normal daylight or artificial light, and only becomes visible by using light of the proper wave length. When appropriately filtered light strikes the fluorescent dye, any tartar, calculus, decay, etc. will glow brightly in its respective color. U.S. Pat. No. 3,624,219 by Perlitsch employs erythrosin as it persists in the mouth to the degree desirable for plaque-disclosing purposes. Block U.S. Pat. Nos. 3,723,613 and 4,064,229 developed a two-tone dye test comprising the combination of the FDC Red #3 (erythrosin) with either FDC Green #3, FDC Blue #1 or Hercules Green Shade 3 in order to obtain differential staining, i.e. thick old plaques stain blue and thin new plaques stain red.

Some synthetic dyes or artificial colorants have been linked to numerous diseases or illnesses, including asthma, thyroid tumors, depression and anxiety, attention deficient disorders, particularly in children. In addition, several synthetic dyes or artificial colorants are thought to be carcinogenic. In fact, several European countries have banned the use of some of these synthetic dyes or artificial colorants. While the research as to the harmful effects for some of these dyes may not be conclusive, the instant invention overcomes the risk of the harmful effect by providing for use of natural colorants.

As used herein, the term “natural colorants” describes colorants, such as lakes, dyes, chemicals, including but not limited to phytochemicals, pigments, derived from or extracted from plants, algae, spices, herbs, or food sources, including but not limited to fruits and vegetables.

Accordingly, a dental disclosing agent used in the instant invention must be capable of adequately penetrating the plaque deposit and stain the plaque so as to be readily visible to the user, without producing an excessively prolonged staining effect. This staining efficacy must be selective so as to identify the areas of plaque-formation on all tooth surfaces and not stain gingival or other oral tissues. This selective staining efficacy must be coupled with easy removability from the mouth by simply brushing, washing, or rinsing after use. In addition, the taste must be pleasant and acceptable to the user, and the color must be pleasing. Lastly, it must be harmless and non-toxic.

The oral composition of the present invention includes a dental disclosing agent delivery composition which is made up of various ingredients, both active and inactive ingredients, which are capable of being mixed together in the form of a toothpaste for delivering the dental disclosing agent to the oral cavity and for providing various teeth cleaning and maintenance functionality. While the oral composition is preferably formulated as a toothpaste, other means of delivery, such as gels or liquids can be formulated.

In some embodiments, the dental disclosing agent delivery composition includes surfactants. Suitable non-limiting representatives of surfactants may include sulfated butyl oleate, medium and long chain fatty acid esters, sodium oleate, salts of fumaric acid, potassium glomate, organic acid esters of mono- and diglycerides, stearyl monoglyceridyl citrate, succistearin, dioctyl sodium sulfosuccinate, glycerol tristearate, lecithin, hydroxylated lecithin, sodium lauryl sulfate, sodium dodecyl sulphate, ammonium lauryl sulfae, acetylated monoglycerides, succinylated monoglycerides, monoglyceride citrate, ethoxylated mono- and diglycerides, sorbitan monostearate, calcium stearyl-2-lactylate, sodium stearyl lactylate, lactylated fatty acid esters of glycerol and propylene glycerol, glycerol-lactoesters of C.sub.8-C.sub.24 fatty acids, polyglycerol esters of C.sub.8-C.sub.24 fatty acids, propylene glycol alginate, sucrose C.sub.8-C.sub.24 fatty acid esters, diacetyl tartaric and citric acid esters of mono- and diglycerides, triacetin, sarcosinate surfactants, isethionate surfactants, tautate surfactants, pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures thereof

In some embodiments, the dental disclosing agent delivery composition includes a fluoride source as fluoride is known to prevent tooth decay and makes teeth stronger as it incorporates itself into teeth enamel. Fluoride compounds have found widespread usage as effective ingredients for inhibiting dental caries. Among those fluoride compounds, fluoride salts which contain stannous ions (e.g. stannous fluoride) have been reported to cause an increase in the fluoride uptake by the dental enamel and consequently in acid-resistance of the enamel after treatment as compared with fluoride salts which do not contain stannous ions. U.S. Pat. No. 3,105,798 discloses a dentifrice composition consisting essentially of a water-soluble fluoride salt, stannous tin and a water-soluble source of six carbon aldonate groups capable of forming water-soluble complexes with stannous tin, the molar ratio of the aldonate group to stannous tin being in the range of from about one:one to about three:one, the molar ratio of stannous tin to fluoride ions being greater than one:one, said dentifrice having a pH of from about 5 to 7. Crystals of sodium pentafluorostannite (NaSn.sub.2 F.sub.5) obtained by reacting one mole of sodium fluoride with two moles of stannous fluoride are described in U.S. Pat. No. 3,490,866. The use of stannous salts of polyphosphonic acids such as methanediphosphonic acid or ethane-1-hydroxy-1, 1-diphosphonic acid described in U.S. Pat. No. 3,549,677. The prior art compositions containing stannous ions and fluoride ions are effective to some extent for dental caries reduction, but their effectiveness is not so extremely high. Moreover, they require repeated application because of their lower reactivity to the tooth surface upon application or readily decreased retention of effectiveness.

Suitable non-limiting representative forms of fluoride include sodium monofluorophosphate, sodium fluoride, and stannous fluoride. Stannous fluoride is commonly incorporated into toothpastes for therapeutic efficacy in the control of dental caries. Stannous fluoride gels, rinses, and dentifrices have since been shown to provide clinical efficacy for the reduction of dental caries, dentinal hypersensitivity, dental plaque and gingivitis. In addition to these clinical effects, formulations containing stannous fluoride may also help to provide improved breath benefits through chemical and antibacterial actions.

In some embodiments, the dental disclosing agent delivery composition includes abrasives. Suitable non-limiting representative abrasives include silicas, aluminas, phosphates, carbonates and combinations thereof. In some embodiments, the abrasive agent is a silica selected from: precipitated silica, silica gels and combinations thereof. Moreover, in some embodiments the abrasive agent is selected from the following: calcium carbonate, sodium bicarbonate, sodium metaphosphate, potassium metaphosphate, tricalcium phosphate, dicalcium phosphate, dehydrated dicalcium phosphate, calcium hydrogen orthophosphate, and combinations thereof.

Hydrated silica is used as a dental abrasive to maximize cleaning and minimizing tooth abrasion. The ability to optimize such characteristics in the past has been limited generally to controlling the structures of the individual components utilized for such purposes for there has been no way of determining how along an individual should be brushing. For instance, if the teeth are clean then the use of a dental abrasive is of no effect toward the cleaning and may actually be harmful. However, if the teeth are unclean then the use of a dental abrasive is necessary to effectively remove the foreign particles. Prior art was limited to improving the dental abrasive and no teaching was directed to determining how much abrasion contact was necessary. The instant invention allows the consumer to visually determine the amount of brushing required, namely the brushing must be continued until all disclosing material is removed. Examples of modifications in precipitated silica structures for such dentifrice purposes are described in the art within such publications as U.S. Pat. Nos. 3,967,563, 3,988,162, 4,420,312, and 4,122,161 to Wason, U.S. Pat. Nos. 4,992,251 and 5,035,879 to Aldcroft et al., U.S. Pat. No. 5,098,695 to Newton et al., and U.S. Pat. Nos. 5,891,421 and 5,419,888 to McGill et al. Modifications in silica gels have also been described within such publications as U.S. Pat. No. 5,647,903 to McGill et al., U.S. Pat. No. 4,303,641, to DeWolf, I I et al., U.S. Pat. No. 4,153,680, to Seybert, and U.S. Pat. No. 3,538,230, to Pader et al. Such disclosures teach improvement in such silica materials in order to impart increased cleaning capacity and reductions in dentin abrasion levels for dentifrice benefits. However, these improvements lack the ability to deliver preferred property levels that accord a dentifrice producer the ability incorporate such an individual material in different amounts with disclosure components in order to effectuate different resultant levels of such cleaning and abrasion characteristics. Silica combinations involving compositions of differing particle sizes and specific surface areas are disclosed in U.S. Pat. No. 3,577,521. to Karlheinz Scheller et al., U.S. Pat. No. 4,618,488 to Macyarea et al., U.S. Pat. No. 5,124,143 to Muhlemann, and U.S. Pat. No. 4,632,826 to Ploger et al. Such resultant dentifrices, however, fail to provide desired levels of abrasion and cleaning simultaneously by use of a visual indicator. The instant invention combines the use of a dental disclosing agent, a fluoride ion, and a dental abrasive within a single paste

In some embodiments, the dental disclosing agent delivery composition includes humectants such as, but not limited to, water, sorbitol, glycerine, xylitol, or combinations thereof.

In some embodiments, the dental disclosing agent delivery composition includes thickeners. Suitable non-limiting representative thickeners include, methyl cellulose, alginates, carrageenan, xanthan gum, gelatin, carob, tragacanth, locust bean, and carboxy methyl cellulose, cellulose gum, acidulants such as malic acid, adipic acid, citric acid, tartaric acid, fumaric acid, and mixtures thereof.

In some embodiments, the dental disclosing agent delivery composition includes preservatives. Suitable non-limiting representative preservatives include sodium benzoate, ethyl paraben, methyl paraben, and combinations thereof.

In some embodiments, the dental disclosing agent delivery composition includes flavoring agents. Suitable non-limiting representatives flavoring those flavors known to one of skill in the art, such as natural and artificial flavors, and include synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, and fruit essences including apple, pear, peach, grape, strawberry, raspberry, cherry, plum, pineapple, apricot, aldehyde flavorings, and combinations thereof.

In some embodiments, the dental disclosing agent delivery composition includes sweeteners. Suitable non-limiting representatives sweeteners include selected from a wide range of materials including water-soluble sweeteners, water-soluble artificial sweeteners, water-soluble sweeteners derived from naturally occurring water-soluble sweeteners, dipeptide based sweeteners, and protein based sweeteners, including mixtures thereof. Illustrative examples include soluble saccharin salts, i.e., sodium or calcium saccharin salts, ihydrochalcones, monellin, steviosides, glycyrrhizin, dihydroflavenol, and sugar alcohols such as sorbitol, mannitol, maltitol, and L-aminodicarboxylic acid aminoalkenoic acid ester amides

In some embodiments, the dental disclosing agent delivery composition includes coloring agents for providing a desired color of the oral composition. Non-limiting representative coloring agents may include titanium dioxide, food colorings and dyes, preferably naturally derived, such as F.D.&C dyes and lakes.

In some embodiments, the dental disclosing agent delivery composition includes vitamins, such as Vitamin E, alpha-tocopherol.

In some embodiments, the dental disclosing agent delivery composition includes antimicrobial agents/antibacterial agents. Suitable non-limiting representative antimicrobial agents/antibacterial agents include triclosan, xylitol, or cetylpyridium chloride employed alone or in combination thereof. The antibacterial agent extends the shelf life of the dental composition but can further minimize the microbial population in the mouth. Since the oral environment is conducive to microbial growth and subject to the reintroduction of food and microorganisms, and because plaque and calculus are continually being deposited on teeth, the composition must address the microbial growth during the cleaning process. Dental plaque which forms on tooth surfaces and restorations are colonies of harmful bacteria, which cannot be flushed away by simply rinsing with water. Active brushing of the teeth is required to remove the adherent plaque and the use of triclosan will destroy the harmful bacteria.

In some embodiments, the dental disclosing agent delivery composition includes chelating agents. Non-limiting chelating agents include pyrophosphates, triphosphates, polyphosphates, polyphosphonates, dialkali metal pyrophosphate salt, a tetra alkali, tetrasodium pyrophosphate, tetrapotassium pyrophosphate, sodium tripolyphosphate, polyphosphate salt, EDTA (ethylenediaminetetraacetate) and salts of EDTA such as calcium disodium ethylenediaminetetraacetate dehydrate, (CaNa₂EDTA.2H₂O, Calcium Disodium EDTA FCC) or disodium ethylenediaminetetraacetate dehydrate (Na₂H₂EDTA.2H₂O, Disodium EDTA FCC, Edetate Disodium USP), and combinations thereof. The use of EDTA, and its salts, either in the dental disclosing agent delivery composition or disclosing agent composition serves several functions. First, it will act as a plaque softening and degrading agent, aiding in the removal of plaque. The actual process would involve chelation of trace metals having multivalent ions, such as iron (Fe), copper (Cu), manganese (Mn), calcium (Ca), magnesium (Mg), or zinc (Zn). In addition, ETDA, and their salts, by chemically binding and effectively chelating the trace metals, minimizes the effects of the trace metals on the color, flavor, and shelf-life capacity.

In some embodiments, the dental disclosing agent delivery composition includes anti-tartar agents such as tetrasodium pyrophosphate.

The following tables are illustrative embodiments of the dental disclosing agent delivery composition and the dental disclosing agent composition. While Table 1 describes the dental disclosing agent delivery composition in the preferred embody form of a toothpaste, the disclosing agent delivery composition may be formulated in other forms, such as, but not limited to, gel or liquid formulations.

TABLE 1
Dental disclosing agent delivery composition:

| | Quantity % |
|---|---|
| Component | w/w or w/v |
| Dicalcium phosphate | 37.0-45.0% |
| (calcium hydrogen orthophosphate) | |
| Water | 5.0-25.0% |
| Sorbitol (D-Glucitol) | 10.0-20.0% |
| Glycerin | 8.0-20.0% |
| Tetrasodium pyrophosphate (TSPP) | 0.25-3.0% |
| Sodium lauryl sulphate | 0.5-2.0% |
| (sodium dodecyl sulphate) | |
| Sodium saccharin | 0.10-2.5% |
| (1,2-Benzisothiazol-3(2H)-one, 1,1-dioxide, | |
| sodium salt) | |
| Titanium dioxide | 0.0-1.5% |
| Dental disclosing agent composition | As needed |
| Sodium benzoate | 0.0-3.0% |
| Cellulose gum (Carboxymethyl cellulose) | 0.5-4.0% |
| Alpha-tocopherol (Vitamin-E Natural) | 0-5.0% |
| Sodium Fluoride (Active ingredient) | 0.1-0.25% |
| Triclosan | 0.0-3.0% |
| (2,4,4′-Trichloro-2′hydroxydiphenyl ether) | |
| calcium disodium ethylenediaminetetraacetate | 0.0-1.7% |
| dehydrate | |
| (CaNa₂EDTA•2H₂0, Calcium Disodium EDTA FCC) | |
| disodium ethylenediaminetetraacetate dehydrate | 0.0-1.73% |
| (Na₂H₂EDTA•2H₂0, Disodium EDTA FCC, Edetate | |
| Disodium USP) | |

Tables 2-4 illustrate several embodiments of the dental disclosing agent composition in accordance with the instant invention.

TABLE 2
Dental disclosing agent composition Example 1:

| | Quantity % |
|---|---|
| Component | w/w or w/v |
| Water | 10.0-40.0% |
| Sodium lauryl sulphate | 0.0-10% |
| (sodium dodecyl sulphate) | |
| calcium disodium ethylenediaminetetraacetate | 0.0-1.62% |
| dehydrate | |
| (CaNa₂EDTA•2H₂0, Calcium Disodium EDTA FCC) | |
| disodium ethylenediaminetetraacetate dehydrate | 0.0-1.62% |
| (Na₂H₂EDTA•2H₂0, Disodium EDTA FCC, Edetate | |
| Disodium USP) | |
| Sodium Benzoate | 0.0-1.30% |
| Red Beet Extract | 10.0-40.0% |
| (extracted from red beetroot, concentrated and | |
| pasteurized by physical means to standardized | |
| color | |
| Lycopene (Extracted from tomatoes, emulsified | 5.0-30.0% |
| and stabilized) | |
| Pomegranate Concentrate (extracted from | 0.0-15.0% |
| pomegranates, concentrated and pasteurized | |
| and/or stabilized by physical means) | |

In addition to extracting lycopene from tomatoes, other sources, such as red carrots, watermelons, papayas, or plants and algae may be used as well.

TABLE 3
Dental disclosing agent composition, Example 2

| | Quantity % |
|---|---|
| Component | w/w or w/v |
| Water | 25.0-50.0% |
| Sodium lauryl sulphate | 0.0-10.0% |
| (sodium dodecyl sulphate) | |
| calcium disodium ethylenediaminetetraacetate | 0.0-1.73% |
| dehydrate | |
| (CaNa₂EDTA•2H₂0, Calcium Disodium EDTA FCC) | |
| disodium ethylenediaminetetraacetate dehydrate | 0.0-1.73% |
| (Na₂H₂EDTA•2H₂0, Disodium EDTA FCC, Edetate | |
| Disodium USP) | |
| Sodium Benzoate | 0.0-1.38% |
| Red Beet Extract | 5.0-35.0% |
| (extracted from red beetroot, concentrated and | |
| pasteurized by physical means to standardized | |
| color | |
| Red Beet Extract Powder | 5.0-35.0% |
| (extracted from red beetroot, mixed with | |
| maltodextrin and spray dried)) | |
| Pomegranate Concentrate (extracted from | 0.0-15.0% |
| pomegranates, concentrated and pasteurized | |
| and/or stabilized by physical means) | |

TABLE 4
Dental disclosing agent composition, Example 3:

| | Quantity % |
|---|---|
| Component | w/w or w/v |
| Water | 10.0-70.0% |
| Sodium lauryl sulphate | 0.0-10% |
| (sodium dodecyl sulphate) | |
| calcium disodium ethylenediaminetetraacetate | 0.0-1.73% |
| dehydrate | |
| (CaNa₂EDTA•2H₂0, Calcium Disodium EDTA FCC) | |
| disodium ethylenediaminetetraacetate dehydrate | 0.0-1.73% |
| (Na₂H₂EDTA•2H₂0, Disodium EDTA FCC, Edetate | |
| Disodium USP) | |
| Sodium Benzoate | 0.0-2.0% |
| Red Beet Extract | 0.0-10.0% |
| (extracted from red beetroot, concentrated and | |
| pasteurized by physical means to standardized | |
| color | |
| Red Beet Extract Powder | 0.0-10.0% |
| (extracted from red beetroot, mixed with | |
| maltodextrin and spray dried)) | |
| Purple Carrot Extract Powder (Extracted from | 15.0-85.0% |
| carrots, clarified, concentrated by physical | |
| means and spray dried) | |

The process for producing the dental disclosing agent delivery composition of the oral composition for identifying plaque located in a person's mouth in accordance with the instant invention is a four phase process:

Phase A: The process begins by mixing the glycerin, sorbital, and cellulose gum. The components are combined using moderate agitation until the liquid is clear ad has no lumps

Phase B: Tetrasodium pyrophosphate, sodium saccharin, sodium fluoride, and sodiumbenzoate is added to water (distilled or RO/DI) at a temperature of 50-60 degrees Celsius. The mixture is mixed with moderate agitation until comments are dissolved (Phase B).

Phase B component is added to Phase A component using elevated agitation. The two components are mixed until clear (lumps).

Phase C: Dicalcium phosphate is added to the Phase A/Phase B mixture using elevated agitation. The Mixture is agitated until uniform. The mixture is then slowly and completely de-aerated.

Phase D: Sodium lauryl sulphate is added to the dental disclosing agent composition, and if used flavoring. The components are mixed using moderate agitation until the mixture is uniform.

The dental disclosing agent composition process occurs in two phases:

Phase 1: Sodium lauryl sulphate, EDTA, and sodium benzoate is added to water at a temperature of 50-60 degrees Celsius. The mixture is mixed until clear.

Phase 2: Desired extracts are added to the Phase 1 mixture and allowed to mix using low agitation. If using a mixture of powder extracts and liquid extracts, powder extracts should be added first. The mixture is allowed to mix until it is clear. No aeration is performed. This mixture is then ready to be added to the dental disclosing agent delivery composition at prescribed quantities at the finished temperature.

### Example

Formulation Ingredient List:

| | *Quantity % |
|---|---|
| *Component Description | Range-w/w |
| Water | q.s. |
| Dicalcium phosphate | 25-45.00% |
| Sorbitol | 15-30% |
| Glycerin | 10-20% |
| Cellulose Gum-CMC Aqualon | 0.5-2.5% |
| Tetra sodium pyrophosphate(TSPP) | 0.25-1.5% |
| Sodium Lauryl Sulphate (30% Sol.) | 0.5-8% |
| Sodium saccharin | 0.1-1.5% |
| Purple Carrot Extract-Dry Powder | 10-28% |
| Sodium benzoate | 0.10-2.5% |
| Versene-NA | 0.1-1.5% |
| Sodium Fluoride | 0.24% |
| Natural & Artificial Flavoring-Mint | 0.05-1.0% |
| Natural Mint Oil | 0-1.0% |
| Pomegranate Concentrate | 0-7.0% |

### Basic Compounding Process Outline

### Phase-A

Mix Glycerin, Sorbitol and CMC, Combine with moderate agitation till clear (no lumps). Hold.

### Phase-B

Add TSPP, Sodium Saccharin, Sodium Fluoride, and Sodium Benzoate and Versene-NA to water (Distilled or RO/DI) at 50-60 C., mix with moderate agitation till dissolved.
Add B into A with elevated agitation, mix till clear (no lumps) to give A/B.

### Phase-C

Add Dicalcium phosphate to A/B with elevated agitation till uniform. Slow and De-aerate completely.

### Phase-D

Add SLS, dye package, and Flavor, mix with moderate agitation till uniform. Hold.

### Basic Compounding Process Outline for Dye Package

There are two (2) phases for all Supplement/Dye packages:
Phase A: Add SLS, Sodium Benzoate and Versene-NA, to water at 40-50 C. mix till clear.
Phase B: Add to A all extracts, mix at low agitation till clear (Do not aerate), to form dye package.
*Add to backbone dye package as directed above at prescribed quantities at finished temp.

It is to be understood that while a certain form of the invention is disclosed, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An oral composition for identifying plaque located in a person's mouth comprising:
a dental disclosing agent composition for revealing the presence of dental plaque in a person's mouth comprising a natural colorant derived from a purple carrot.

2. The oral composition for identifying plaque located in a person's mouth according to claim 1 wherein said natural colorant is a purple carrot extract.

3. The oral composition for identifying plaque located in a person's mouth according to claim 2 wherein said purple carrot extract is prepared by spray dying.

4. The oral composition for identifying plaque located in a person's mouth according to claim 2 wherein the purple carrot extract has an approximately equal content of carbohydrate and sugar.

5. The oral composition for identifying plaque located in a person's mouth according to claim 2 wherein a 1% solution (by weight) of the natural colorant as a starting material has an absorption of 11.5-12.5 at a wave-length of 425.

6. The oral composition for identifying plaque located in a person's mouth according to claim 1 wherein said dental disclosing agent composition further comprises red beet extract.

7. The oral composition for identifying plaque located in a person's mouth according to claim 7 wherein said dental disclosing agent delivery composition further comprises, lycopene.

8. The oral composition for identifying plaque located in a person's mouth according to claims 1-7, further comprising one or more of surfactants, a fluoride source, abrasives, humectants, thickeners, preservatives, antimicrobial agents, flavoring agents, sweeteners, vitamins, and coloring agents.

9. The oral composition for identifying plaque located in a person's mouth according to claim 8 wherein said oral composition comprises sodium fluoride.

10. The oral composition for identifying plaque located in a person's mouth according to claim 8 wherein said oral composition comprises triclosan.

11. The oral composition for identifying plaque located in a person's mouth according to claim 8 wherein said oral composition comprises at least one chelating agent.

12. The oral composition for identifying plaque located in a person's mouth according to claim 11 wherein said at least one chelating agent is selected from the group consisting of calcium disodium ethylenediaminetetraacetate dehydrate and disodium ethylenediaminetetraacetate dehydrate.

13. The oral composition for identifying plaque located in a person's mouth according to claim 8 wherein said oral composition comprises a flavoring.

14. The oral composition for identifying plaque located in a person's mouth according to claim 13 wherein said flavorings are derived from natural sources.

15. The oral composition for identifying plaque located in a person's mouth according to claim 1 wherein said dental disclosing agent composition further comprises pomegranate concentrate.

16. An oral composition for identifying plaque located in a person's mouth comprising:
a dental disclosing agent composition for revealing the presence of dental plaque in a person's mouth comprising a combination of natural colorants derived from a plurality of fruit or vegetable sources, said fruit or vegetable sources including a purple carrot extract;
a dental disclosing agent delivery composition for delivering said dental disclosing agent to a person's mouth, said delivery composition comprising dicalcium phosphate in a concentration of about 37.0% to about 45.0%, water in a concentration of about 5.0% to about 25.0%, sorbitol in a concentration of about 10.0% to about 20.0%, glycerin in a concentration of about 8.0% to about 20.0%, tetrasodium pyrophosphate in a concentration of about 0.25% to about 3.0%, sodium lauryl sulphate in a concentration of about 0.5% to about 2.0%, sodium saccharin in a concentration of about 0.10% to about 2.5%, titanium dioxide in a concentration of about 0.0% to about 1.5%, sodium benzoatein a concentration of about 0.0% to about 3.0%, cellulose gum in a concentration of about 0.5% to about 4.0%, alpha-tocopherol in a concentration of about 0.1% to about 0.0% to about 5.0%, sodium fluoride in a concentration of about 0.1% to about 0.25%, triclosan in a concentration of about 0.0% to about 3.0%, calcium disodium ethylenediaminetetraacetate dehydrate in a concentration of about 0.0% to about 1.5%, and disodium ethylenediaminetetraacetate dehydrate a concentration of about 0.0% to about 1.5%.

17. The oral composition for identifying plaque located in a person's mouth according to claim 16 wherein said dental disclosing agent composition comprises water in a concentration of about 10.0% to about 40.0%, sodium lauryl sulphate in a concentration of about 0.0% to about 10.0%, calcium disodium ethylenediaminetetraacetate dehydrate in a concentration of about 0.0% to about 1.62%, disodium ethylenediaminetetraacetate dehydrate in a concentration of about 0.0% to about 1.62%, sodium benzoate in a concentration of about 0.0% to about 1.3%, red beet extract in a concentration of about 10.0% to about 40.0%, and pomegranate concentrate in a concentration of about 0.1 to about 15.0%.

18. The oral composition for identifying plaque located in a person's mouth according to claim 16 wherein said dental disclosing agent composition comprises water in a concentration of about 25.0% to about 50.0%, sodium lauryl sulphate in a concentration of about 0.0% to about 10.0%, calcium disodium ethylenediaminetetraacetate dehydrate in a concentration of about 0.0% to about 1.73%, disodium ethylenediaminetetraacetate dehydrate in a concentration of about 0.0% to about 1.73%, sodium benzoate in a concentration of about 0.0% to about 1.38%, and pomegranate concentrate in a concentration of about 0.1 to about 15.0%.

19. The oral composition for identifying plaque located in a person's mouth according to claim 16 wherein said dental disclosing agent composition comprises water in a concentration of about 10.0% to about 70.0%, sodium lauryl sulphate in a concentration of about 0.0% to about 10.0%, calcium disodium ethylenediaminetetraacetate dehydrate in a concentration of about 0.0% to about 1.73%, disodium ethylenediaminetetraacetate dehydrate in a concentration of about 0.0% to about 1.73%, sodium benzoate in a concentration of about 0.0% to about 2.0%, and pomegranate concentrate in a concentration of about 0.1 to about 15.0%.
